(19) United States
(12) Patent Application Publication
(10) Pub. No.: US 2021/0284713 A1
(43) Pub. Date: Sep. 16, 2021

(54) ANTI COVID-19 THERAPIES USING CD40 LIGAND FUSION PROTEIN

(71) Applicant: ImmunityBio, Inc., Culver City, CA (US)

(72) Inventors: Kayvan Niazi, Culver City, CA (US); Jay Gardner Nelson, Culver City, CA (US); Annie Shin, Culver City, CA (US); Clifford Anders Olson, Culver City, CA (US); Shiho Tanaka, Culver City, CA (US)

(21) Appl. No.: 17/082,994

(22) Filed: Oct. 28, 2020

Related U.S. Application Data
(60) Provisional application No. 62/991,504, filed on Mar. 18, 2020.
(60) Provisional application No. 62/988,328, filed on Mar. 11, 2020.

(51) Int. Cl. C07K 14/705; C07K 14/005; C12N 15/86; A61K 39/215

(52) U.S. Cl. CPC C07K 14/70575; C07K 14/005; A61K 2039/54; A61K 39/215; C12N 15/86

(57) ABSTRACT

Compositions and methods are presented for prevention and/or treatment of a coronavirus disease wherein the composition comprises a recombinant entity. The recombinant entity comprises a nucleic acid that encodes a extracellular portion of CD40 ligand (CD40L) coupled by a flexible linker to a coronavirus 2 (CoV2) spike protein and/or a CoV2 nucleocapsid protein.

This application claims priority to our copending U.S. provisional patent applications with the Ser. No. 62/988,328, filed Mar. 11, 2020; 62/991,504 filed on Mar. 18, 2020; 63/009,960 filed Apr. 14, 2020; 63/010,010 filed Apr. 14, 2020; 63/016,048 filed Apr. 27, 2020; 63/016,241 filed Apr. 27, 2020; 63/022,146 filed May 8, 2020; 63/053,691 filed Jul. 19, 2020, and 63/059,975 filed Aug. 1, 2020. This application further claims priority to our copending U.S. utility application Ser. No. 16/880,804 filed May 21, 2020, and Ser. No. 16/883,263 filed May 26, 2020, Each of these applications are incorporated by reference in its entirety.

### SEQUENCE LISTING

The content of the ASCII text file of the sequence listing named Sequences_102538.0081US_ST25, which is 92 KB in size was created on Aug. 20, 2020 and electronically submitted via EFS-Web along with the present application. The sequence listing is incorporated by reference in its entirety.

### FIELD

The present disclosure relates to composition, systems, and methods of treating subjects diagnosed or suspected to have Coronavirus Disease 2019 (COVID-19).

### BACKGROUND

The background description includes information that may be useful in understanding the present disclosure. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

After several noteworthy coronavirus outbreaks in the recent years, including SARS and MERS, COVID-19 is yet another example of a serious infectious disease precipitated by a member of the corona virus family. COVID-19 patients with severe symptoms are treated to maintain respiration/blood oxygenation. Despite such interventions, the mortality rate is significant, particularly in elderly, immune compromised individuals, and individuals with heart disease, lung disease, or diabetes.

All known methods of treating COVID-19 suffer from various disadvantages. Consequently, there is a need for improved compositions and methods that provide therapeutic effect, that reduce or prevent viral entry into a cell, reduce direct and indirect toxicity of the virus to the patient, and that produce an effective immune response.

### SUMMARY

The present disclosure describes various immune therapeutic compositions and methods for treating and/or preventing a coronavirus disease. In one aspect, disclosed herein is an expression cassette comprising a promoter operably linked to a recombinant nucleic acid that encodes a CD40 ligand (CD40L) extracellular portion coupled by a flexible linker to a coronavirus 2 (CoV2) spike protein and/or a CoV2 nucleocapsid protein. In one embodiment, the flexible linker has between 4 and 50 amino acids, and optionally comprises a (GS)x sequence. In preferred embodiments, the recombinant nucleic acid encodes a fusion protein having at least 85% identity to any one or more of SEQ ID NOs:6-11.

In another aspect, disclosed herein is a replication defective adenovirus comprising: an E1 gene region deletion; an E2b gene region deletion; and a nucleic acid encoding a promoter operably linked to a recombinant nucleic acid that encodes a CD40L extracellular portion coupled by a flexible linker to a CoV2 spike protein and/or a CoV2 nucleocapsid protein. In one embodiment, the CoV2 nucleocapsid protein has at least 85% identity to SEQ ID NO:1. In one embodiment, the CoV2 nucleocapsid protein is fused to an endosomal targeting sequence (N-ETSD) having at least 85% identity to SEQ ID NO:2. In one embodiment, the CoV2 spike protein has at least 85% identity to SEQ ID NO:4 or SEQ ID NO:5. In one embodiment, the recombinant nucleic acid encodes a fusion protein having at least 85% identity to any one or more of SEQ ID NOs:6-11.

In certain embodiments, the adenovirus described above may further comprise a nucleic acid encoding a trafficking sequence, a co-stimulatory molecule, and/or an immune stimulatory cytokine. In one embodiment, the co-stimulatory molecule is selected from the group consisting of CD80, CD86, CD30, CD40, CD30L, ICOS-L, B7-H3, B7-H4, CD70, OX40L, 4-1BBL, GITR-L, TIM-3, TIM-4, CD48, CD58, TL1A, ICAM-1, and LFA3, and wherein the immune stimulatory cytokine is selected from the group consisting of IL-2, IL-12, IL-15, IL-21, IPS1, and LMP1.

In one embodiment, disclosed herein is a vaccine composition comprising the adenovirus described above, wherein the composition is formulated for injection. In one embodiment, disclosed herein is a method for inducing immunity against CoV2 in a patient in need thereof, the method comprising administering the vaccine composition to the patient.

In yet another aspect of this disclosure, provided herein is a recombinant yeast comprising a nucleic acid encoding a promoter operably linked to a recombinant nucleic acid that encodes a CD40L extracellular portion coupled by a flexible linker to a CoV2spike protein and/or a CoV2 nucleocapsid protein. In certain embodiments, the recombinant yeast is Saccharomyces cerevisiae. In certain embodiments, the CoV2 nucleocapsid protein has at least 85% identity to SEQ ID NO:1. In certain embodiments, the CoV2 nucleocapsid protein is fused to an N-ETSD having at least 85% identity to SEQ ID NO:2. In certain embodiments, the CoV2 spike protein has at least 85% identity to SEQ ID NO:4 or SEQ ID NO:5. In certain embodiments, the recombinant nucleic acid encodes a fusion protein having at least 85% identity to SEQ ID NOs:6-11.

In certain embodiments, the recombinant yeast described above further comprises a nucleic acid encoding a trafficking sequence, a co-stimulatory molecule, and/or an immune stimulatory cytokine. The co-stimulatory molecule may be selected from the group consisting of CD80, CD86, CD30, CD30L, ICOS-L, B7-H3, B7-H4, CD70, OX40L, 4-1BBL, GITR-L, TIM-3, TIM-4, CD48, CD58, TL1A, ICAM-1, and LFA3. The immune stimulatory cytokine may be selected from the group consisting of IL-2, IL-12, IL-15, IL-21, IPS1, and LMP1.

In one embodiment a vaccine composition is disclosed, comprising the recombinant yeast as described above, wherein the composition is formulated for injection. In one embodiment, disclosed herein is a method for inducing immunity against CoV2 in a patient in need thereof, the method comprising administering the vaccine composition to the patient.

Throughout the disclosure, the preferred coronavirus disease is COVID-19.

Various objects, features, aspects, and advantages will become more apparent from the following detailed description of preferred embodiments, along with the accompanying drawing in which like numerals represent like components.

### BRIEF DESCRIPTION OF THE DRAWING

FIG. 1 depicts transfection of 293T and B16F10 with CD40LRF plasmids.

FIG. 2 depicts expression of TNFα following AdV hCD40LRF infection of human GM-DCs.

FIG. 3 depicts expansion of T-cells with or without hCD40LRF.

### DETAILED DESCRIPTION

Immune response against CoV2 can be enhanced or dampened by interference with CD40 signaling in antigen presenting cells (APCs). Immune response against a CoV2 virus can be significantly enhanced by inducing APCs to express one or more antigens associated with the CoV2 virus. Vaccine compositions that induce expression of a chimeric protein comprising CD40 ligand and a CoV2 associated antigen can treat patients infected with CoV2 virus. Thus, recombinant expression cassettes are described herein that include nucleic acid sequences encoding CD40L protein and a CoV2 associated antigen. Most preferably, the CoV2 associated antigen is CoV2 spike protein or CoV2 nucleocapsid protein.

In one aspect, the present disclosure provides an expression cassette comprising a promoter operably linked to a recombinant nucleic acid that encodes a extracellular portion of CD40L, for example CD40L ectodomain, coupled by a flexible linker to a CoV2spike protein and/or a CoV2 nucleocapsid protein. In certain embodiments, the flexible linker has between 4 and 50 amino acids, and optionally comprises a (GnS)x sequence. As further disclosed herein, the expression cassette may be placed in an expression vector. The expression vector may be (e.g.) a bacterial expression vector, a viral expression vector, or a yeast expression vector.

In a second aspect, disclosed herein are recombinant viruses and yeasts. The viruses and yeasts disclosed herein may be useful for a variety of purposes, such as treating and/or preventing a coronavirus disease.

In one embodiment of this aspect, the expression cassette disclosed above may be placed in a virus. The virus is preferably a replication defective adenovirus, with an E1 gene region deletion and an E2b gene region deletion. The virus also comprises nucleotides encoding CD40L chimeric protein fused to a CoV2 spike protein and/or CoV2 nucleocapsid protein.

In certain embodiments, the expression cassette disclosed above may be placed in a recombinant yeast. Preferably, the recombinant yeast is S. cerevisiae. The recombinant yeast may comprise nucleic acid sequences encoding CD40L protein fused to a CoV2 spike protein and/or CoV2 nucleocapsid protein.

In certain embodiments, the CoV2 nucleocapsid protein comprises a sequence with at least 80% (e.g., at least 85%, at least 90%, at least 95%, at least 98%, or at least 99%) identity to SEQ ID NO:1.

In certain embodiments, the CoV2 nucleocapsid protein is fused to N-ETSD. Any intracellular antigen can be driven to cell surface expression by tagging the antigen with ETSD as described herein. In one embodiment, the N-ETSD comprises a sequence with at least 80% (e.g., at least 85%, at least 90%, at least 95%, at least 98%, or at least 99%) identity to SEQ ID NO:2. In certain embodiments, there may be a linker between the N-ETSD domain and the nucleocapsid protein. For example this linker may be a 16 amino acid linker having the sequence (GGGS)₄. In certain embodiments, the fusion protein comprising N-ETSD and CoV2 nucleocapsid protein may be encoded by a nucleic acid sequence according to SEQ ID NO:3.

The CoV2 spike protein may have at least 80% (e.g., at least 85%, at least 90%, at least 95%, at least 98%, or at least 99%) identity to SEQ ID NO:4. The nucleic acid encoding the CoV2 spike protein can be SEQ ID NO:5.

In one embodiment, the recombinant fusion protein disclosed herein is preferably at least 80% (e.g., at least 85%, at least 90%, at least 95%, at least 98%, or at least 99%) identical to any one or more of SEQ ID NOs:6-11.

SEQ ID NO:6 (S-WT-hCD40L) comprises, in a sequential manner, a native leader peptide, CoV2 Spike ectodomain, GGGS×4 (glycine-serine linker), & human CD40L ectodomain.

SEQ ID NO:7 (S-HA-hCD40L) comprises, in a sequential manner, native leader peptide, HA tag, CoV2 Spike ectodomain, glycine-serine linker, & human CD40L ectodomain.

SEQ ID NO:8 (S-HA-GS-hCD40L) comprises, in a sequential manner, native leader peptide, HA tag, glycine-serine linker, CoV2 Spike ectodomain, fibritin trimerization domain, glycine-serine linker, & human CD40L ectodomain.

SEQ ID NO:9 (S-WT-FTD-hCD40L) comprises, in a sequential manner, native leader peptide, CoV2 Spike ectodomain, fibritin trimerization domain, glycine-serine linker, & human CD40L ectodomain.

SEQ ID NO:10 (S-HA-FTD-hCD40L) comprises, in a sequential manner, native leader peptide, HA tag, CoV2 Spike ectodomain, fibritin trimerization domain, glycine-serine linker, & human CD40L ectodomain.

SEQ ID NO:11 (S-HA-GS-FTD-hCD40L) comprises, in a sequential manner, native leader peptide, HA tag, glycine-serine linker, CoV2 Spike ectodomain, fibritin trimerization domain, glycine-serine linker, & human CD40L ectodomain.

The expression cassettes, adenoviruses and yeasts disclosed herein may further comprise a nucleic acid encoding a trafficking sequence, a co-stimulatory molecule, and/or an immune stimulatory cytokine. The co-stimulatory molecule may be selected from the group consisting of CD80, CD86, CD30, CD40, CD30L, ICOS-L, B7-H3, B7-H4, CD70, OX40L, 4-1BBL, GITR-L, TIM-3, TIM-4, CD48, CD58, TL1A, ICAM-1, & LFA3. The immune stimulatory cytokine may be selected from the group consisting of IL-2, IL-12, IL-15, IL-15 super agonist (nogapendekin alfa-imbakicept), IL-21, IPS1, & LMP1. Additionally, or alternatively, the vaccines disclosed herein may also encode SARS-CoV-2 M protein, with or without an ETSD tag.

All of the above noted co-stimulatory genes are known in the art, and sequence information of these genes, isoforms, and variants can be retrieved from various public resources, including sequence data bases accessible at the NCBI, EMBL, GenBank, RefSeq, etc. Moreover, while the above exemplary stimulating molecules are preferably expressed in full length form as expressed in human, modified and non-human forms (e.g., muteins, truncated forms and chimeric forms) are also suitable so long as such forms assist in stimulating or activating T-cells.

In yet another embodiment, disclosed herein is a vaccine composition comprising the adenovirus or yeast as disclosed above. The vaccine composition is formulated for injection. The vaccine composition may be used for inducing immunity against CoV2 in a patient in need thereof, by administering the vaccine composition to the patient.

Also disclosed herein are methods for preventing and/or treating coronavirus diseases, especially COVID-19. Preferably, the method includes administering a viral or yeast vector that encodes the nucleocapsid protein and/or spike protein of the coronavirus in an immunogenic composition. The virus and/or yeast vaccine, thus administered, innoculates the individual with CoV2 nucleocapsid or spike protein. The individual then gains an immune response against CoV2 nucleocapsid or spike protein. Coronaviral nucleocapsid and spike proteins are relatively conserved polypeptides, so immune responses can be elicited for a variety of coronaviruses.

An adenoviral vector may be modified to encode the fusion protein comprising CD40L and CoV2 nucleocapsid protein or spike protein. Similarly, a yeast vector may also be modified to encode the fusion protein comprising CD40L and the CoV2 nucleocapsid protein or spike protein.

In one embodiment, the present disclosure provides a vaccine formulation comprising a recombinant entity, wherein the recombinant entity comprises a nucleic acid encoding CD40L and the CoV2 nucleocapsid protein or spike protein. The vaccine formulation may be useful for treating a disease, such as a coronavirus mediated disease or infection.

### Coronaviral Nucleocapsid & Spike Proteins

Coronaviruses cause a variety of diseases in humans, such as the Severe Acute Respiratory Syndrome (SARS) that spread to several countries in 2002-2003. Another such disease is COVID-19.

Coronavirus virions are spherical to pleomorphic enveloped particles. The envelope is studded with projecting glycoproteins, and surrounds a core consisting of matrix protein enclosed within which is a single strand of positive-sense RNA. The terms “nucleocapsid protein,” “nucleoprotein,” and “nucleocapsid” are used interchangeably herein. The nucleocapsid (N) is a structural protein found in all coronaviruses.

Spike (S) protein is also found throughout all coronaviruses. S mediates fusion between the viral membrane and the host cellular membrane.

### CD40L

CD40L is a TNF family ligand (preferably an extracellular portion of a ligand). A fusion protein having an extracellular portion of CD40L and the extracellular portion of a spike or nucleocapsid protein can stimulate a B cell or T cell mediated response. The CD40L-S or CD40L-N fusion protein can interact with CD40, leading to B and T cell activation and proliferation.

The CD40L-S or CD40L-N fusion protein activates CD40 and transmits CD40-mediated signal into APCs as if it had been contacted by another cell expressing CD40L (e.g., CD4+ T cell). CD40, like many other TNF family members, needs to trimerize to effect signaling. Trimerization results from CD40's interaction with the CD40L trimerization domain.

All CD40L variants are suitable for use herein. Human and other mammalian CD40Ls are particularly suitable. Numerous such sequences are known (see e.g., uniprot sequence database). In certain non-limiting embodiments, the CD40L includes its native signal peptide, although other signal peptides may also be included or substituted. CD40L should retain its trimerization domain for activating chimeric constructs. On the other hand, where down-regulation is desired, the CD40L may have a truncated trimerization domain or some other sufficient steric hindrance to disrupt trimerization.

Most typically, CD40L will APC species (e.g., human CD40 for human APC, etc.). In certain embodiments, the trimerization domain may be optimized to increase affinity, or may be partially or entirely deleted. In still further examples, one or more amino acids may be exchanged (especially at the N-terminus) to increase half-life.

Suitable linkers enable mobility between the CD40L and S or N portions to permit selective binding. For activating chimeric molecules, the linker will have between 4 and 60 amino acids, with low or no immunogenicity. Suitable linkers include GS-type linkers with between 8 and 50, or between 4 and 25, and most preferably between 15 and 17 amino acids. There are numerous alternative linkers known (see e.g., Adv Drug Deliv Rev (2013) 65(10):1357-69), and all of them are suitable for use herein.

### Expression Cassettes

Recombinant expression cassettes encoding the chimeric proteins described above can include a first nucleic acid segment encoding CD40L portion (an extracellular domain of CD40L and optionally a leader peptide coupled to the N-terminus of the extracellular domain of CD40L) and CoV2 S or N portions in a single reading frame, so that the CD40L portion and the CoV2 S or N portion can be encoded in a single polypeptide. Where the leader peptide is to be coupled with the CD40L extracellular domain, the nucleic acid segment encoding the leader peptide can be in the same reading frame with the segment encoding the CD40L extracellular domain, with or without a linker in between. Fusion proteins may include intervening sequences (e.g., 2A sequences, or a glycine-serine linker) or may be direct fusions. Expression cassettes include a promoter (constitutive or inducible) to drive expression of the sequences encoding the chimeric proteins. As the chimeric protein has a transmembrane portion, the chimera will typically have a signal sequence (optionally cleavable) to direct the chimera to the cell surface.

### Additional Molecules Encoded by the Recombinant Expression Cassette

Additionally, the recombinant expression cassette may further comprise a third nucleic acid segment encoding one or more co-stimulatory molecules and/or cytokines to modulate immune response. Suitable co-stimulatory molecules include B7.1 (CD80), B7.2 (CD86), CD30L, CD40, CD48, CD70, CD112, CD155, ICOS-L, 4-1BB, GITR-L, LIGHT, TIM3, TIM4, ICAM-1, LFA3 (CD58), and members of the SLAM family. Suitable cytokines include immune stimulatory cytokines (e.g., IL-2, IL-15, IL-17, IL-21, etc.) for increasing immune response, or a down-regulating cytokine (e.g., IL-10, TGF-β, etc.) to dampen immune response. Alternatively, or additionally, the nucleic acid further may also include a sequence encoding at least one component of a SMAC (e.g., CD2, CD4, CD8, CD28, Lck, Fyn, LFA-1, CD43, and/or CD45 or their respective binding counterparts). In certain embodiments, the nucleic acid may additionally comprise a sequence encoding a STING pathway activator, such as a chimeric protein in which a transmembrane domain of LMP1 of EBV is fused to a signaling domain of IPS-1.

In one preferred embodiment, the cytokine is an IL-15 superagonist (IL-15N72D), and/or an IL-15 superagonist/IL-15Rα Sushi-Fc fusion complex (e.g., nogapendik alfa-imbakicept) coupled with at least one of IL-7, IL-15, IL-18, IL-21, & IL-22, or preferably both IL-7 and IL-21. Any suitable variations of IL-15 superagonists are contemplated.

### Expression Vectors

Most typically, the recombinant expression cassette is placed in an expression vector, such that the nucleic acid segment encoding the peptide can persist through cell divisions. For example, the recombinant expression cassette is a DNA/RNA fragment, and suitable expression vectors are linear or circular DNA/RNA constructs. A preferred expression vector is a nonreplicating recombinant adenovirus genome, optionally with a deleted or non-functional E1 and/or E2b gene.

In still further embodiments, the expression vector can be a bacterial vector that can be expressed in a genetically-engineered bacterium, which expresses endotoxins at a level low enough not to cause an endotoxic response in human cells and/or insufficient to induce a CD-14 mediated sepsis when introduced to the human body. Suitable bacteria include ClearColi® BL21(DE3) electrocompetent cells. This strain is BL21 with a genotype F⁻ ompT hsdSB (rB⁻ mB⁻) gal dcm ion λ(DE3 [lacI lacUV5-T7 gene 1 ind1 sam7 nin5]) msbA148 ΔgutQΔkdsD ΔlpxLΔlpxMΔpagPΔlpxPΔeptA. Several specific deletion mutations (ΔgutQ ΔkdsD ΔlpxL ΔlpxMΔpagPΔlpxPΔeptA) encode the modification of LPS to Lipid IVA, while one additional compensating mutation (msbA148) enables the cells to maintain viability in the presence of IVA. These mutations delete the oligosaccharide chain from the LPS, more specifically, two of the six acyl chains. While electrocompetent BL21 bacteria are provided as an example, the genetically modified bacteria can be also chemically competent bacteria.

Alternatively or additionally, the expression vector can be a yeast vector that can be expressed in yeast. Preferred yeast includes S. cerevisiae (e.g., GI-400 series recombinant immunotherapeutic yeast strains, etc).

The recombinant nucleic acids described herein need not be limited to viral, yeast, or bacterial expression vectors. Suitable vectors also include DNA vaccine vectors, linearized DNA, and mRNA, all of which can be transfected into suitable cells following known protocols.

### Recombinant Viruses

All known manners of making recombinant viruses are suitable for use herein. Especially preferred viruses are those already established in therapy, including adenoviruses, adeno-associated viruses, alphaviruses, herpes viruses, lentiviruses, etc. Adenoviruses are particularly preferred.

Preferably the virus is replication deficient and non-immunogenic. For example, suitable viruses include genetically modified alphaviruses, adenoviruses, adeno-associated viruses, herpes viruses, lentiviruses, etc. For example, genetically modified replication defective adenoviruses are preferred that are suitable not only for multiple vaccinations but also vaccinations in individuals with preexisting immunity to the adenovirus (see e.g., WO 2009/006479 and WO 2014/031178, which are incorporated by reference in their entireties). In certain embodiments, the replication defective adenovirus vector comprises a replication defective adenovirus 5 vector. In certain embodiments, the replication defective adenovirus vector comprises a deletion in the E2b region. In certain embodiments, the replication defective adenovirus vector further comprises a deletion in the E1 region. In that regard, deletion of the E2b gene and other late proteins in the genetically modified replication defective adenovirus reduces immunogenicity. Such genetically modified viruses allow for relatively large recombinant cargo. High titers of these recombinant viruses can be achieved using modified human 293 cells (e.g., J Virol. (1998) 72(2): 926-33).

In a further embodiment, the adenovirus vectors contemplated for use in the present disclosure include adenovirus vectors that have a deletion in the E2b region of the Ad genome and, optionally, deletions in the E1, E3 and, also optionally, partial or complete removal of the E4 regions. In a further embodiment, the adenovirus vectors for use herein have the E1 and/or the preterminal protein functions of the E2b region deleted. In some cases, such vectors have no other deletions. In another embodiment, the adenovirus vectors for use herein have the E1, DNA polymerase, and/or the preterminal protein functions deleted.

“E2b deleted”, as used herein, refers to a specific DNA sequence that is mutated in such a way so as to prevent expression and/or function of at least one E2b gene product. Thus, in certain embodiments, “E2b deleted” is used in relation to a specific DNA sequence that is deleted (removed) from the Ad genome. E2b deleted or “containing a deletion within the E2b region” refers to a deletion of at least one base pair within the E2b region of the Ad genome. Thus, in certain embodiments, more than one base pair is deleted and in further embodiments, at least 20, 30, 40, 50, 60, 70, 80, 90, 100, 110, 120, 130, 140, or 150 base pairs are deleted. In another embodiment, the deletion is of more than 150, 160, 170, 180, 190, 200, 250, or 300 base pairs within the E2b region of the Ad genome. An E2b deletion may prevent expression and/or function of at least one E2b gene product. “E2b deletion” encompasses deletions within exons of encoding portions of E2b-specific proteins as well as deletions within promoter and leader sequences. In certain embodiments, an E2b deletion prevents expression and/or function of one or both of the DNA polymerase and the preterminal protein of the E2b region. In a further embodiment, “E2b deleted” refers to one or more point mutations in the DNA sequence of this region of an Ad genome such that one or more encoded proteins is non-functional. Such mutations include residues that are replaced with a different residue leading to a change in the amino acid sequence that result in a nonfunctional protein.

Compositions and methods presented are not only suitable for directing virally expressed antigens specifically to one or another (or both) MHC systems, but will also provide increased stimulatory effect on the CD8+ and/or CD4+ cells via inclusion of various co-stimulatory molecules (e.g., ICAM-1 (CD54), ICOS-L, LFA-3 (CD58), and at least one of B7.1 (CD80) and B7.2 (CD86)), and via secretion or membrane bound presentation of checkpoint inhibitors.

All therapeutic recombinant viral expression systems are suitable for use herein so long as such viruses are capable to lead to expression of the recombinant payload in an infected cell. Regardless of the type of recombinant virus, the virus may infect patient (or non-patient) cells ex vivo or in vivo. For example, the virus may be injected subcutaneously or intravenously, or may be administered intranasally or via inhalation to so infect the patient's cells, and especially antigen presenting cells. Alternatively, immune competent cells (e.g., NK cells, T cells, macrophages, dendritic cells, etc.) of the patient (or from an allogeneic source) may be infected in vitro and then transfused to the patient. Alternatively, immune therapy need not rely on a virus but may be effected with nucleic acid transfection or vaccination using RNA or DNA, or other recombinant vector that leads to the expression of the neoepitopes (e.g., as single peptides, tandem mini-gene, etc.) in desired cells, and especially immune competent cells.

Nucleic acid sequences for expression from virus infected cells are under the control of appropriate, known regulatory elements. For example, suitable promoter elements include constitutive strong promoters (e.g., SV40, CMV, UBC, EF1A, PGK, CAGG promoter), but inducible promoters are also suitable. Exemplary inducible promoters include those sensitive to hypoxia and promoters sensitive to TGF-β or IL-8 (e.g., via TRAF, JNK, Erk, or other responsive elements promoter). In other examples, suitable inducible promoters include the tetracycline-inducible promoter, the myxovirus resistance 1 (Mx1) promoter, etc.

The replication defective adenovirus comprising an E1 gene region deletion, an E2b gene region deletion, and a nucleic acid encoding a CoV2 N and/or S protein may be administered to a patient in need for inducing immunity against CoV2. Routes and frequency of administration of the therapeutic compositions described herein, as well as dosage, may vary from individual to individual, and the severity of the disease, and may be readily established using standard techniques. In certain embodiments, the administration comprises delivering 4.8-5.2×10¹¹ replication defective adenovirus particles, or 4.9-5.1×10¹¹ replication defective adenovirus particles, or 4.95-5.05×10¹¹ replication defective adenovirus particles, or 4.99-5.01×10¹¹ replication defective adenovirus particles.

Administration of virus particles can be through a variety of suitable paths for delivery. One preferred route is injection, such as intracutaneous injection, intramuscular injection, intravenous injection, or subcutaneous injection.

### Recombinant Yeasts

All known yeast strains are suitable for use herein. However, Saccharomyces strains are preferred. In one aspect of any of the embodiments of the disclosure described above or elsewhere herein, the yeast vehicle is a whole yeast. The whole yeast, in one aspect is killed. In one aspect, the whole yeast is heat inactivated. In one preferred embodiment, the yeast is a whole, heat-inactivated S. cerevisiae. By way of example, WO 12/109404 discloses yeast for treatment of chronic hepatitis B infection.

Any yeast strain can be used to produce a yeast vehicle of the present disclosure. One consideration for the selection of yeast for use as an immune modulator is the pathogenicity of the yeast. In preferred embodiments, the yeast is non-pathogenic, such as S. cerevisiae. Non-pathogenic yeast strains minimize any adverse effects to the individual to whom the yeast vehicle is administered. However, pathogenic yeast may also be used if the pathogenicity of the yeast can be negated using pharmaceutical intervention.

Suitable exemplary yeast genera include Saccharomyces, Candida, Cryptococcus, Hansenula, Kluyveromyces, Pichia, Rhodotorula, Schizosaccharomyces, and Yarrowia. In one aspect, yeast genera are selected from Saccharomyces, Candida, Hansenula, Pichia or Schizosaccharomyces, and in a preferred aspect, Saccharomyces is used. Species of yeast strains that may be used include Saccharomyces cerevisiae, Saccharomyces carlsbergensis, Candida albicans, Candida kefyr, Candida tropicalis, Cryptococcus laurentii, Cryptococcus neoformans, Hansenula anomala, Hansenula polymorpha, Kluyveromyces fragilis, Kluyveromyces lactis, Kluyveromyces marxianus var. lactis, Pichia pastoris, Rhodotorula rubra, Schizosaccharomyces pombe, and Yarrowia hpolytica.

A number of these species include a variety of subspecies, types, subtypes, etc. that are intended to be included within the aforementioned species. In one aspect, yeast species used in the instant disclosure include S. cerevisiae, C. albicans, H. polymorpha, P. pastoris, and S. pombe. S. cerevisiae is useful for its relative ease to manipulate and for being “Generally Recognized As Safe” or “GRAS” for use as a food additive (GRAS, FDA proposed Rule 62FR18938, 17 Apr. 1997). Yeast strains capable of replicating plasmids to a particularly high copy number include the S. cerevisiae cir strain, which is capable of supporting expression vectors that allow one or more target antigen(s) and/or antigen fusion protein(s) and/or other proteins to be expressed at high levels. In addition, any mutant yeast strains can be used, including those that exhibit reduced post-translational modifications of expressed target antigens or other proteins, such as mutations in the enzymes that extend N-linked glycosylation.

Protein expression in yeast can be accomplished using techniques known to those skilled in the art. Most typically, a nucleic acid molecule encoding at least one protein is inserted into an expression vector in such manner that the nucleic acid molecule is expressed when transformed into a host yeast cell. Nucleic acid molecules encoding one or more proteins can be on one or more expression vectors operatively linked to one or more expression control sequences. Particularly important expression control sequences are those which control transcription initiation, such as promoter and upstream activation sequences.

Any suitable yeast promoter can be used in the methods and compositions of the present disclosure. A variety of such promoters are known to those skilled in the art and have generally be discussed above. Promoters for expression in S. cerevisiae include promoters of yeast genes encoding the following: alcohol dehydrogenase I (ADH1) or II (ADH2), CUP1, phosphoglycerate kinase (PGK), triose phosphate isomerase (TPI), translational elongation factor EF-1α (TEF2), glyceraldehyde-3-phosphate dehydrogenase (GAPDH), galactokinase (GAL1), galactose-1-phosphate uridyl-transferase (GAL7), UDP-galactose epimerase (GAL10), cytochrome c1 (CYC1), Sec7 protein (SECT) and acid phosphatase (PHO5), including hybrid promoters such as ADH2/GAPDH and CYC1/GAL10 promoters, and including the ADH2/GAPDH promoter, which is induced when glucose concentrations in the cell are low (e.g., about 0.1-0.2%), as well as the CUP1 promoter and the TEF2 promoter. Likewise, a number of upstream activation sequences (UASs), also referred to as enhancers, are known. Upstream activation sequences for expression in S. cerevisiae include the UASs of genes encoding the following proteins: PCK1, TPI, TDH3, CYC1, ADH1, ADH2, SUC2, GAL1, GAL7 and GAL10, as well as other UASs activated by the GAL4 gene product, with the ADH2 UAS being used in one aspect. Since the ADH2 UAS is activated by the ADR1 gene product, it may be preferable to overexpress the ADR1 gene when a heterologous gene is operatively linked to the ADH2 UAS. Transcription termination sequences for expression in S. cerevisiae include the termination sequences of the alpha-factor, GAPDH, and CYC1 genes. Transcription control sequences to express genes in methyltrophic yeast include the transcription control regions of the genes encoding alcohol oxidase and formate dehydrogenase.

Likewise, transfection of a nucleic acid molecule into a yeast cell can be accomplished by any method by which a nucleic acid molecule administered into the cell and includes diffusion, active transport, bath sonication, electroporation, microinjection, lipofection, adsorption, and protoplast fusion. Transfected nucleic acid molecules can be integrated into a yeast chromosome or maintained on extrachromosomal vectors using known techniques. As discussed above, yeast cytoplast, yeast ghost, and yeast membrane particles or cell wall preparations can also be produced recombinantly by transfecting intact yeast microorganisms or yeast spheroplasts with desired nucleic acid molecules, producing the antigen therein, and then further manipulating the microorganisms or spheroplasts using known techniques to produce cytoplast, ghost, or subcellular yeast membrane extract or fractions thereof containing desired antigens or other proteins. Further exemplary yeast expression systems, methods, and conditions suitable for use herein are described in US 2010/0196411, 2012/0107347, 2017/0224794, and 2017/0246276.

### Administration

Recombinant viruses and yeasts as described above may be individually or in combination used as a therapeutic vaccine. Recombinant nucleic acids (or recombinant expression cassettes) and/or the recombinant virus carrying the recombinant nucleic acids can be used to induce or generate antigen presenting cells (e.g., dendritic cells) in vivo or ex vivo. The chimeric proteins and CoV2 nucleocapsid and/or spike proteins produced can enhance anti-coronavirus immune response. One or more recombinant viruses including one or more nucleic acid segments encoding the chimeric protein and/or one or more CoV2 associated antigen (such as N or S), cytokine, and/or co-stimulatory molecule can be administered to patient APCs in vivo. Such infected APCs express one or more CoV2 nucleocapsid and/or spike proteins, cytokines, and/or co-stimulatory molecules to stimulate immune response against the coronavirus cells.

For example, a genetically modified virus carrying the recombinant nucleic acid encoding the chimeric protein and/or CoV2 nucleocapsid and/or CoV2 spike proteins can be formulated in any pharmaceutically acceptable carrier (e.g., preferably formulated as a sterile injectable composition, etc.) to form a pharmaceutical composition. The sterile composition can be administered in any suitable methods. In certain embodiments, where a cytokine (e.g., N-803) is to be expressed in the same cell, the recombinant nucleic acid further includes a nucleic acid encoding the cytokine. Additionally or alternatively, another recombinant virus (or bacteria or yeast) can be co-administered including a recombinant nucleic acid encoding the cytokine. Where two or more types of the recombinant virus are desired to infect the same antigen presenting cell, the two or more types of the recombinant virus can be formulated in a single pharmaceutical composition. However, the two or more types of the recombinant virus can also be formulated in two separate and distinct pharmaceutical compositions and administered to the patient concurrently or substantially concurrently (e.g., within an hour, within 2 hours, etc.)

Where the pharmaceutical composition includes the recombinant virus, the titer should be between 10⁴ and 10¹² virus particles per dosage unit. All known routes and modes of administration are suitable for use herein. Where the pharmaceutical composition includes recombinant bacteria, the titer should be between 10² and 10³, between 10³ and 10⁴, or between 10⁴ and 10⁵ colony forming units (cfu) per dosage unit. Where the pharmaceutical composition includes recombinant yeast, the titer should be between 10² and 10³, between 10³ and 10⁴, or between 10⁴ and 10⁵ cfus per dosage unit.

As used herein, “administering” a virus, bacteria, or yeast formulation refers to both direct and indirect administration. Direct administration is typically performed by a health care professional (e.g., physician, nurse, etc.). Indirect administration includes providing or making available the formulation to the health care professional for direct administration (e.g., via injection, infusion, oral delivery, topical delivery, etc.).

In certain embodiments, the virus, bacterial or yeast formulation is injected systemically, including subcutaneous, subdermal, or intravenous injection.

Dose and/or schedule of administration may vary depending on depending on the type of virus, bacteria, or yeast, prognosis of disease, and health status of the patient (e.g., including age, gender, etc.). While it may vary, the dose and schedule may be selected and regulated so that the formulation has little significant toxic effect to normal host cells, yet sufficient to elicit an immune response. Thus, in a preferred embodiment, an optimal administration can be determined based on a predetermined threshold. For example, the predetermined threshold may be a predetermined local or systemic concentration of specific type of cytokine (e.g., IFN-γ, TNF-β, IL-2, IL-4, IL-10, etc.). Dose, route, and schedule are typically adjusted to have immune response-specific cytokines expressed at least 20%, at least 30%, at least 50%, at least 60%, at least 70% more at least locally or systemically.

For example, where the pharmaceutical composition includes recombinant virus, the dose is at least 10⁶ virus particles/day, or at least 10⁸ virus particles/day, or at least 10¹⁰ virus particles/day, or at least 10¹¹ virus particles/day. In certain embodiments, a single dose of virus formulation can be administered at least once a day or twice a day (half dose per administration) for at least a day, at least 3 days, at least a week, at least 2 weeks, or at least a month. In other embodiments, the dose of the virus formulation can be gradually increased during the schedule, or gradually decreased during the schedule. In still other embodiments, several series of formulations can be administered, each separated by an interval (e.g., one administration each for 3 consecutive days and one administration each for another 3 consecutive days with an interval of 7 days, etc.).

In certain embodiments, the formulation can be administered in two or more stages: e.g, a priming administration and a boost administration. The priming dose can be higher than the following boosts (e.g., at least 20% higher, preferably at least 40%, more preferably at least 60%, etc.). Alternatively, the priming dose can be lower than the following boosts. Additionally, where there is a plurality of boosts, each boost can have a different dose (e.g., increasing dose, decreasing dose, etc.).

Embodiments of the present disclosure are further described in the following examples. The examples are merely illustrative and do not in any way limit the scope of the invention as claimed.

### Example 1: Transfection of 293T and B16F10

A recombinant plasmid DNA encodes the human CD40L with cytomegalovirus (CMV) pp-65 (“CD40LRF”). Once infected, the human body retains CMV antigens for life. PP-65 is often used as a model system in the development of new therapeutics, and especially new vaccines. A DNA plasmid comprising the CD40L and pp-65 would function similar to a DNA plasmid comprising the CD40L and CoV2 nucleocapsid or spike protein.

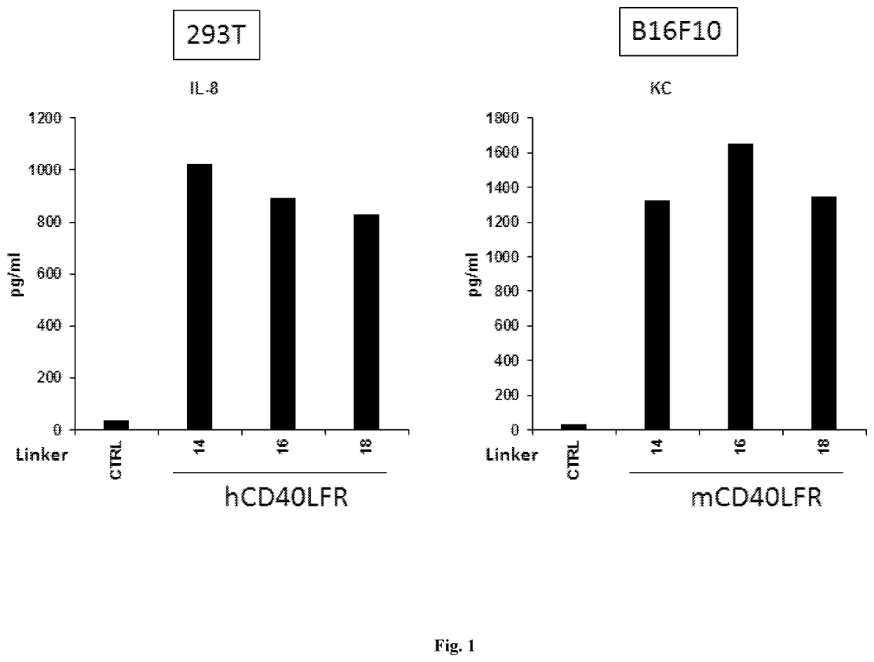

FIG. 1 illustrates the results of transfection of 293T (kidney cells) and B16F10 (melanoma cells) with CD40LRF plasmids. To whether the linker length between the CD40L and the pp-65 fragment would have any effect, CD40LRF plasmids were constructed with three different linker lengths, having 14 amino acids, 16 amino acids, and 18 amino acids respectively. As a control, cultures of 293T and B16F10 were transfected with an empty vector plasmid.

The IL-8 or KC (CXCR2) output was recorded in each case. Both 293T and B16F10 transfections showed a strong T cell output regardless of linker length.

### Example 2: Expression of TNFα Following AdV hCD40LRF Infection of Human GM-DCs

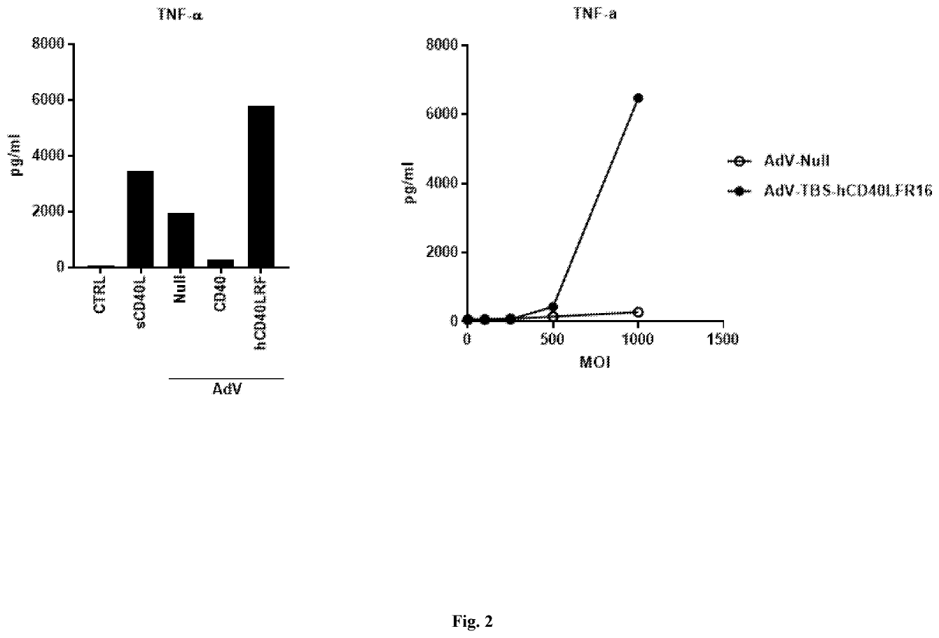

Human dendritic cells were infected with a replication defective adenovirus having E1 and E2b gene deletions, and further having CD40LRF expression constructs. TNFα expression was measured. The left-most column in FIG. 2A depicts TNFα secretion from T cells in contact with dendritic cells (negative control). The second column depicts TNFα secretion from T cells contacted with a soluble form of CD40L (sCD40L, positive control). The third column depicts TNFα secretion when a human dendritic cell was infected with an empty adenovirus (without CD40L or pp-65). The fourth column depicts TNFα secretion when a human dendritic cell was infected with an adenovirus encoding CD40L, but not pp-65. Finally, the fifth column depicts TNFα secretion when a human dendritic cell was infected with an adenovirus encoding CD40L and pp-65. FIG. 2A shows that TNFα expression is highest following AdV hCD40LRF (adenovirus encoding CD40L and pp-65) infection of human GM-DCs. The adenovirus encoding CD40L and CoV2 spike protein or nucleocapsid protein is expected to function like the adenovirus encoding CD40L and pp-65, and is also expected to provide a high expression of TNFα when such adenovirus is infected in dendritic cells.

FIG. 2B illustrates a titration of TNFα expression by controlling the multiplicity of infection (MOI). Once the MOI exceeds 500, TNFα expression following infection with an adenovirus encoding CD40L and pp-65 increases in a linear manner, but stays constant in the null adenovirus.

### Example 3: Expansion of T-Cells with or without hCD40LRF

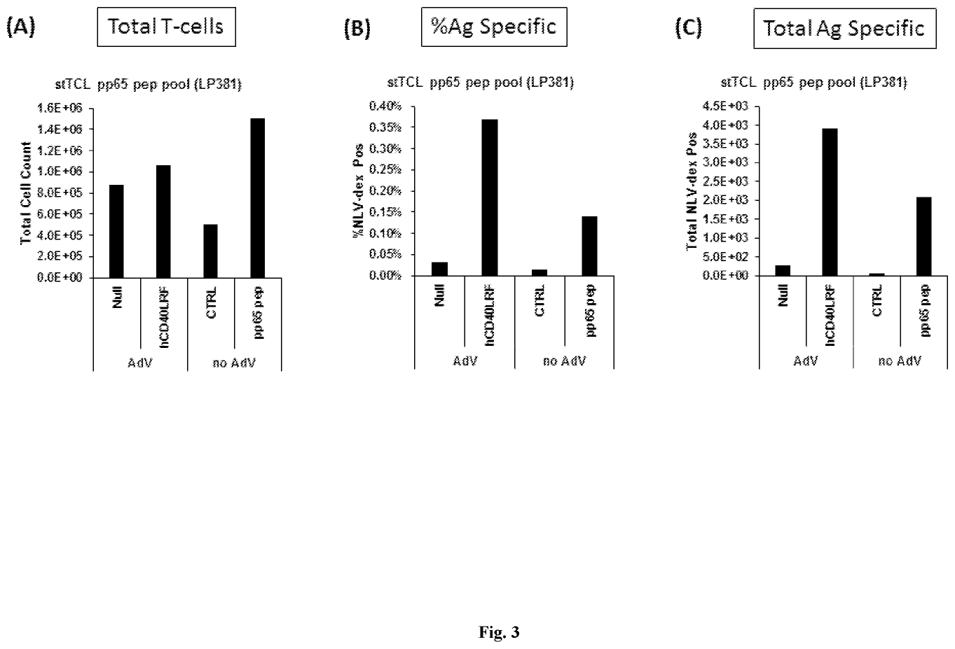

FIG. 3A illustrates total T cell count upon transfection with the hCD40LRF construct and adenoviral transduction of the construct. The total T cell populations are similar in both cases, indicating that the immune system is not overworked or inflated.

FIG. 3B illustrates the percentage of antigen specific T cells expanding after transfection with the hCD40LRF construct and adenoviral transduction of the construct. A higher percentage of T cell expansion followed adenoviral transduction than followed transfection.

FIG. 3C illustrates total antigen specific T cells expanding after transfection with the hCD40LRF construct and adenoviral transduction of the construct. A higher T cell expansion total followed adenoviral transduction than followed transfection.

### Example 4: Track Record of Rapid Vaccine Development Utilizing Second Generation Human (hAd5) Adenovirus Platform During Pandemic Treats: H1N1 Experience in 2009

Vaccines against emerging pathogens such as the 2009 H1N1 pandemic virus can benefit from current technologies such as rapid genomic sequencing to construct the most biologically relevant vaccine. The hAd5 [E1−, E2b−, E3−] platform induces immune responses to various antigenic targets. This vector platform expressed hemagglutinin (HA) and neuraminidase (NA) genes from 2009 H1N1 pandemic viruses. Inserts were consensuses sequences designed from viral isolate sequences and the vaccine was rapidly constructed and produced. Vaccination induced H1N1 immune responses in mice, which afforded protection from lethal virus challenge. In ferrets, vaccination protected from disease development and significantly reduced viral titers in nasal washes. H1N1 cell mediated immunity as well as antibody induction correlated with the prevention of disease symptoms and reduction of virus replication. The hAd5 [E1−, E2b−, E3−] has thus demonstrated the capability for the rapid development of effective vaccines against infectious diseases.

### Example 6: Rationale for Inclusion of Nucleocapsid (N) in hAd5 Constructs for COVID-19

The SARS-CoV-2 N protein is highly conserved and highly expressed. Previous research with the related coronavirus that causes SARS demonstrated that N protein is immunogenic (Gupta, 2006), when integrated with intracellular trafficking constructs. To date, vaccine strategies in development all involve developing immunogenicity against S protein. However, very recent evidence in patients who recovered from COVID-19 demonstrates anti-N Th1 immunity (Grifoni, 2020). A second report by Grifoni et al. further confirmed that in the predictive bioinformatics model, T and B cell epitopes were highest for both S & N (Grifoni, 2020). The present disclosure confirms the potential that combining S with N, that long-term cell-mediated immunity with a Th1 phenotype can be induced. The potential exists for this combination vaccine to serve as a long-term “universal” COVID-19 vaccine in light of mutations undergoing in S and the finding that the structural N protein is highly conserved in the coronavirus family. The clinical trial is designed to compare S alone versus S+N, to demonstrate safety and to better inform the immunogenicity of S and S+N. A single construct having S & N would be selected to induce potent humoral and cell mediated immunity.

In one aspect of any of the embodiments described above or elsewhere herein, the composition is formulated in a pharmaceutically acceptable excipient suitable for administration to a subject.

The immunotherapeutic compositions disclosed herein may be either “prophylactic” or “therapeutic.” When provided prophylactically, compositions are provided in advance of the development of, or the detection of the development of, a coronavirus disease, to prevent, inhibit, or delay the development of the coronavirus disease. Prophylactic compositions can be administered to individuals that appear to be coronavirus disease free (healthy, or normal, individuals), or to individuals who have not yet been diagnosed with coronavirus. Individuals who are at high risk for developing a coronavirus disease, may be treated prophylactically with a composition of the instant disclosure.

When provided therapeutically, the immunotherapy compositions are provided to an individual who is diagnosed with a coronavirus disease, to ameliorate or cure the coronavirus disease.

The recitation of ranges of values herein serves as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the disclosures herein, and does not pose a limitation on the scope of the invention otherwise claimed. No language in the specification should be construed as indicating any non-claimed element essential to the practice of the claimed invention.

Many more modifications besides those already described are possible without departing from the concepts disclosed herein. Where the specification claims refer to at least one of something selected from the group consisting of A, B, C . . . and N, the text should be interpreted as requiring only one element from the group, not A plus N, or B plus N, etc.

What is claimed is:

1. An expression cassette comprising a promoter operably linked to a recombinant nucleic acid that encodes a extracellular portion of CD40 ligand (CD40L) coupled by a flexible linker to a coronavirus 2 (CoV2) spike protein and/or a CoV2 nucleocapsid protein.

2. The expression cassette of claim 1, wherein the flexible linker has between 4 and 50 amino acids, and optionally comprises a (GnS)x sequence.

3. The expression cassette of claim 1, wherein the recombinant nucleic acid encodes a fusion protein having at least 85% identity to SEQ ID NO:6, SEQ ID NO:7, SEQ ID NO:8, SEQ ID NO:9, SEQ ID NO:10, or SEQ ID NO:11.

4. A replication defective adenovirus, comprising:
an E1 gene region deletion;
an E2b gene region deletion; and
a nucleic acid encoding a promoter operably coupled to a recombinant nucleic acid that encodes a extracellular portion of CD40 ligand (CD40L) coupled by a flexible linker to a coronavirus 2 (CoV2) spike protein and/or a CoV2 nucleocapsid protein.

5. The adenovirus of claim 4, wherein the CoV2 nucleocapsid protein has at least 85% identity to SEQ ID NO:1.

6. The adenovirus of claim 5, wherein the CoV2 nucleocapsid protein is fused to an endosomal targeting sequence (N-ETSD) having at least 85% identity to SEQ ID NO:2.

7. The adenovirus of claim 4, wherein the CoV2 spike protein has at least 85% identity to SEQ ID NO:4 or SEQ ID NO:5.

8. The adenovirus of claim 7, wherein the recombinant nucleic acid encodes a fusion protein having at least 85% identity to any one or more of SEQ ID NOs:6-11.

9. The adenovirus of claim 4, wherein the adenovirus further comprises a nucleic acid encoding a trafficking sequence, a co-stimulatory molecule, and/or an immune stimulatory cytokine.

10. The adenovirus of claim 9, wherein the co-stimulatory molecule is selected from the group consisting of CD80, CD86, CD30, CD30L, ICOS-L, B7-H3, B7-H4, CD70, OX40L, 4-1BBL, GITR-L, TIM-3, TIM-4, CD48, CD58, TL1A, ICAM-1, and LFA3, and wherein the immune stimulatory cytokine is selected from the group consisting of IL-2, IL-12, IL-15, IL-21, IPS1, and LMP1.

11. A vaccine composition comprising the adenovirus of claim 4, wherein the composition is formulated for injection.

12. A method for inducing immunity against CoV2 in a patient in need thereof, the method comprising administering to the patient the vaccine composition of claim 11.

13. A recombinant yeast comprising a nucleic acid encoding a promoter operably linked to a recombinant nucleic acid that encodes a extracellular portion of CD40 ligand (CD40L) coupled by a flexible linker to a coronavirus 2 (CoV2) spike protein and/or a CoV2 nucleocapsid protein.

14. The recombinant yeast of claim 13, wherein the CoV2 nucleocapsid protein has at least 85% identity to SEQ ID NO:1.

15. The recombinant yeast of claim 14, wherein the CoV2 nucleocapsid protein is fused to an endosomal targeting sequence (N-ETSD) having at least 85% identity to SEQ ID NO:2.

16. The recombinant yeast of claim 13, wherein the CoV2 spike protein has at least 85% identity to SEQ ID NO:4 or SEQ ID NO:5.

17. The recombinant yeast of claim 16, wherein the recombinant nucleic acid encodes a fusion protein having at least 85% identity to any one or more of SEQ ID NOs:6-11.

18. The recombinant yeast of claim 13, wherein the recombinant yeast is Saccharomyces cerevisiae.

19. A vaccine composition comprising the recombinant yeast of claim 13, wherein the composition is formulated for injection.

20. A method for inducing immunity against CoV2 in a patient in need thereof, the method comprising administering to the patient the vaccine composition of claim 19.


SEQUENCE LISTING (11 sequences)

SEQ ID NO: 1 - protein, 473 aa, Artificial Sequence
  Nucleocapsid protein
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAGPGPGNLVPMVATVGPGPGMLIPIAVGGALAGLVLIVLIAYLIGKKHCSYQDIL

SEQ ID NO: 2 - protein, 27 aa, Artificial Sequence
  N-ETSD
MLLLPFQLLAVLFPGGNSEDYKDDDDK

SEQ ID NO: 3 - DNA, 1552 bp, Artificial Sequence
  ETSD
aatgctgctgctgcccttccagttgctggctgtcctctttcccggcggcaactccgaggattacaaggacgacgacgacaagggtggaggctctggaggtggctctggtggaggttccggtggcggatctatgagcgacaacggtccccagaatcaaagaaatgcgcccagaattacattcggcggcccttctgatagcactggctcaaatcaaaacggggagagaagcggagccaggtccaaacagcggagaccccaaggcctgcctaataacaccgcttcctggttcacagctctgacgcaacacggcaaggaggatctgaagtttccacggggtcagggcgtcccgattaacacgaactctagcccagatgaccaaatagggtactacagaagagcgacaaggcggatcagaggaggcgatggaaaaatgaaggatctgtcccctaggtggtatttctattacctgggcacaggccctgaagctgggttgccttacggcgcaaacaaagatggaattatatgggtggccaccgagggggcgttgaacaccccaaaggatcacatcggaacgaggaatcccgccaacaatgctgctatagtgctccaactgccacagggaacaaccctgcctaagggcttctacgccgaggggagccgcggtggcagccaggccagctccagaagttcctcccgcagccggaacagctctagaaacagcactcccggcagctccagagggacaagcccagccagaatggccggcaatggcggcgacgctgccctcgcacttctgttgcttgatcggctcaatcaactcgaaagcaaaatgtccggcaagggacaacaacagcaaggacagaccgttacaaaaaaaagcgccgccgaggctagcaagaagcccagacagaagcgaaccgcaacaaaggcctataatgtaacacaagcctttggaaggcggggacccgaacagacccagggaaattttggcgaccaggaactgatccggcaagggacagactataaacattggccacagatagcgcaatttgctccctccgcctccgccttctttggcatgtcaagaataggcatggaagtaactccttctggaacctggctgacgtacactggggcaatcaagttggatgataaggaccctaatttcaaggaccaagttattttgctcaacaagcatatagacgcctacaagactttcccgcctaccgaacctaaaaaggataagaagaagaaagcagacgagacccaggccctgcctcaacggcaaaagaagcagcaaactgtgacactcctgcccgccgctgacttggatgatttttcaaaacagctccaacagagtatgagcagcgccgatagcacccaagctggaccgggtccgggcaacctggtgccgatggtggcgaccgtgggtccaggaccgggtatgctgatccccatcgccgtgggcggggccctggccggcctcgtgctgatcgtccttatcgcctacctcatcggcaagaagcactgctcatatcaggacatcctgtga

SEQ ID NO: 4 - protein, 1282 aa, Artificial Sequence
  spike protein
MFVFLVLLPLVSSYPYDVPDYAQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 5 - DNA, 3850 bp, Artificial Sequence
  HA-spike
aatgttcgtttttctcgttctcctcccgcttgtgagcagctatccgtatgatgtgccggattatgcgcaatgtgtcaacctcaccacaaggacacagctccctcccgcatatacgaatagctttaccagaggcgtatactatcctgataaggtctttaggagctcagtactgcatagcactcaggatctcttcctgccgttcttcagtaatgttacttggtttcacgccattcatgtttccgggaccaatggcaccaaacggttcgataatccagtgcttcccttcaacgatggggtgtactttgccagcactgaaaaatctaatataattcggggatggattttcggaaccacactcgattccaagactcagtccctcttgatcgttaacaacgctactaatgttgtcattaaggtgtgtgagtttcagttctgcaacgaccctttcctgggtgtctactaccataaaaataacaagagctggatggagtccgaatttcgcgtctactcaagcgccaataattgcacttttgagtatgtgtcccagccctttttgatggatctggagggaaagcagggcaatttcaaaaatctgagagaattcgtttttaagaatatagatggatacttcaaaatctacagcaaacacacacccataaatcttgtgcgcgatcttccccagggcttcagcgcgttggaaccccttgttgacttgcccataggcatcaacattaccaggttccaaacgctgctcgccctccaccgcagctacttgacacccggggattccagctccggatggaccgccggcgccgcagcgtattatgtggggtacctgcaacccaggacatttttgctcaagtacaatgagaatgggaccatcacagatgcggtagactgtgcactggatccactcagcgaaactaaatgtaccctgaaaagctttaccgtggagaaaggaatctaccaaaccagcaacttcagggtccagcccactgaatccatcgttagatttccaaatataactaatttgtgtccatttggagaggtgttcaatgctacaaggttcgcgtctgtatacgcttggaaccggaagcgcatctcaaattgcgtggctgattatagcgttctttacaacagcgcttccttttccacgttcaagtgctatggtgtatccccgacaaagctgaatgacttgtgcttcaccaatgtgtatgcggattctttcgttattcgaggcgatgaagtcagacaaattgcgcctggccagaccggaaagattgccgactacaactataaactgccggacgactttactggttgcgtgatcgcttggaacagcaataatcttgatagtaaagttggaggaaactacaattacctctatagactgttcagaaagagcaacttgaagccattcgaacgggatatctctacggagatctatcaagctggcagcaccccctgcaatggtgtggaaggctttaattgttattttcctttgcagagctatggcttccaacctaccaacggagtgggctaccagccctacagagtggtggtgctcagctttgaactgctgcatgccccggccacagtttgcgggcccaaaaaaagcacgaatctggttaagaacaaatgcgtcaacttcaattttaatgggttgacaggtacaggcgtactgaccgaatccaacaaaaagttcctgccttttcagcagttcgggagagatatcgccgacactacagacgccgtcagggatccccaaacactcgaaattctggacatcacaccttgttccttcggcggggtatctgtgattactccgggcacaaataccagtaaccaggtagcggtgctttaccaggatgtcaactgtacggaagtacctgtcgctattcatgcggatcaactcactcctacctggagagtttattccactgggtccaacgtgtttcagacccgagccggctgcttgattggcgcggaacatgttaacaactcctacgaatgtgacatccctatcggagctggcatctgtgcttcctatcaaacgcaaacgaacagcccacggcgggccagatccgtagcctctcaaagcatcatcgcttatactatgtccttgggggctgaaaacagcgttgcctattccaacaatagcatcgctatccctaccaactttaccatttccgtgaccacagaaatactgccggtgagcatgacaaagacttctgtggactgtaccatgtatatatgcggcgatagcacagagtgttctaatttgctgctgcagtacggcagcttttgtacccaactcaacagagcacttacagggattgccgtcgagcaggataaaaacacccaggaggttttcgcccaggttaagcagatctacaagaccccaccaatcaaggatttcggcggcttcaatttttcccagatactgcccgatccttccaagccatccaaaaggagctttatagaggatctgctgttcaacaaggtgactctggccgacgctggctttatcaagcaatatggcgattgcctgggggatattgccgctagggaccttatctgcgctcaaaaattcaacggtcttaccgttctcccgcccctgctcaccgacgagatgatagcccagtacacgagcgcacttttggccggcacgataaccagcggctggacattcggtgccggggccgctcttcaaatcccctttgccatgcagatggcctacagatttaatgggataggcgtgacacaaaatgtcttgtatgaaaatcagaaactgattgcaaaccagtttaatagcgctattggcaagatccaagatagcctttcctccaccgcatccgctctgggaaagttgcaagacgtcgtgaatcaaaacgcccaagctctgaataccctcgtgaagcagcttagctccaactttggcgcgatatcctccgtgctgaacgatatcctgtccagattggacaaggtcgaggcagaagtccagatcgatagattgataaccggcagactccagtctctgcagacatatgtgactcagcagttgataagagcggccgaaatacgagcgtctgcaaatctcgcagcaacgaaaatgtcagagtgtgtattggggcaaagtaaaagagtagatttctgtggaaagggttaccatctgatgtcattcccccagtctgcaccacatggagtagtttttttgcatgtgacttatgtgcctgcccaggagaaaaatttcaccactgcacctgcgatctgtcatgacggcaaggcacatttccctagagaaggcgtcttcgtatcaaatggaacacactggtttgtaacccaaaggaacttttacgagccccaaattataactaccgacaacaccttcgtaagcggaaactgcgacgtcgttatagggatagtcaataatacggtctatgaccctcttcagccggaactggactcctttaaagaagaactggataagtacttcaagaaccatacgtctccggatgtggatctcggagatataagtggaatcaacgcaagcgtagtaaacattcagaaggagatagaccgactcaatgaggttgctaaaaacctgaacgaaagcttgatagacttgcaggagctgggtaagtacgaacagtacattaagtggccatggtatatctggttgggcttcatagcaggactcatagctatcgtcatggtgacaataatgctttgttgtatgaccagctgttgttcttgtctgaaaggctgctgcagctgtggcagctgttgtaaatttgacgaagatgattccgagcctgtgcttaagggcgtaaaactccactatacatga

SEQ ID NO: 6 - protein, 1294 aa, Artificial Sequence
  S-WT-hCD40L
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDGSGGGNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 7 - protein, 1303 aa, Artificial Sequence
  S-HA-hCD40L
MFVFLVLLPLVSSYPYDVPDYAQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDGSGGGNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 8 - protein, 1319 aa, Artificial Sequence
  S-HA-GS-hCD40L
MFVFLVLLPLVSSYPYDVPDYAGGGSGGGSGGGSGGGSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDGSGGGNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 9 - protein, 1391 aa, Artificial Sequence
  S-WT-FTD-hCD40L
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGGSGGMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 10 - protein, 1400 aa, Artificial Sequence
  S-HA-FTD-hCD40L
MFVFLVLLPLVSSYPYDVPDYAQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGGSGGMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 11 - protein, 1416 aa, Artificial Sequence
  S-HA-GS-FTD-hCD40L
MFVFLVLLPLVSSYPYDVPDYAGGGSGGGSGGGSGGGSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGGSGGMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL